(19) United States
(12) Patent Application Publication
(10) Pub. No.: US 2024/0065369 A1
(43) Pub. Date: Feb. 29, 2024

(54) SYSTEM AND METHOD FOR DETERMINING REGIONAL SENSOR DATA

(71) Applicant: Orpyx Medical Technologies Inc., Calgary (CA)

(72) Inventors: VINCENT CYPRIEN CASTONGUAY-SIU, CALGARY (CA); SANJAY GUPTA, BEDFORD, MA (US)

(21) Appl. No.: 18/449,958

(22) Filed: Aug. 15, 2023

Related U.S. Application Data
(60) Provisional application No. 63/400,113, filed on Aug. 23, 2022.

(51) Int. Cl. A43B 3/44; A61B 5/00; A61B 5/103

(52) U.S. Cl. CPC A43B 3/44; A61B 5/1036; A61B 5/6807

(57) ABSTRACT

A system, method and computer program product for determining regional sensor data. Sensor readings are obtained from a corresponding plurality of sensors. The sensors are grouped into a plurality of sensor regions. Each sensor region includes at least one region-associated sensor from the plurality of sensors. A plurality of regional sensor values are determined for the plurality of sensor regions by, for each sensor region: identifying a region-specific model, the region-specific model specifying how the at least one regional sensor value for that sensor region is to be calculated; and generating the at least one regional sensor value for that sensor region by applying the region-specific model to the sensor readings obtained from the at least one region-associated sensor corresponding to that sensor region. The regional sensor values can account for sensor and conductivity failures and allow the measurement resolution of the sensing unit to be adjusted.

### CROSS-REFERENCE TO RELATED APPLICATIONS

This application claims the benefit of priority of U.S. Provisional Application No. 63/400,113 filed on Aug. 23, 2022, which is incorporated herein by reference.

### FIELD

This document relates to systems and methods for processing data from sensors monitoring human movement or human activity. In particular, this document relates to determining regional sensor data for sensors monitoring human movement or human activity.

### BACKGROUND

U.S. Pat. No. 9,836,165 B2 (Nho et al.) discloses an integrated Silicon-OLED display and touch sensor panel. The integrated Silicon-OLED display and touch sensor panel can include a Silicon substrate, an array of transistors, one or more metallization layers, one or more vias, an OLED stack, color filters, touch sensors, and additional components and circuitry. Additional components and circuitry can include an electrostatic discharge device, a light shielding, a switching matrix, one or more photodiodes, a near-infrared detector and near-infrared color filters. The integrated Silicon-OLED display and touch sensor panel can be further configured for near-field imaging, optically-assisted touch, and fingerprint detection. In some examples, a plurality of touch sensors and/or display pixels can be grouped into clusters, and the clusters can be coupled to a switching matrix for dynamic change of touch and/or display granularity.

United States Patent Application Publication No. 2021/0315485A1 (Matusik et al.) discloses systems and methods for estimating 3D poses of a subject based on tactile interactions with the ground. Test subject interactions with the ground are recorded using a sensor system along with reference information (e.g., synchronized video information) for use in correlating tactile information with specific 3D poses, e.g., by training a neural network based on the reference information. Then, tactile information received in response to a given subject interacting with the ground can be used to estimate the 3D pose of the given subject directly, i.e., without reference to corresponding reference information. Certain exemplary embodiments use a sensor system in the form of a pressure sensing carpet or mat, although other types of sensor systems using pressure or other sensors can be used in various alternative embodiments.

### SUMMARY

The following summary is intended to introduce the reader to various aspects of the detailed description, but not to define or delimit any invention.

A system, method and computer program product for determining regional sensor data is provided. More particularly, in some examples, a plurality of sensors can be grouped into a plurality of sensor regions. Each sensor region can be associated with a corresponding model that is used to generate one or more sensor values for that sensor region. Sensor readings obtained from the plurality of sensors can be used as inputs to the corresponding region-specific models in order to generate the one or more sensor values for the respective sensor regions. The grouping of sensors into sensor regions and the models selected for the plurality of sensor regions can be defined and/or adjusted to provide various advantages. For example, the sensor regions and/or region-specific models can be selected to compensate for one or more failed sensors. The sensor regions and/or region-specific models can also be selected based on a desired measurement resolution for the sensors and/or based on a particular use of the sensors.

According to some aspects, the present disclosure provides a method of determining regional sensor data. The method includes obtaining a plurality of sensor readings from a corresponding plurality of sensors, the plurality of sensors arranged in a first predetermined pattern, wherein the first predetermined pattern maps each of the plurality of sensors to respective locations on a carrier device and the plurality of sensors are grouped into a plurality of sensor regions, wherein each sensor region includes at least one region-associated sensor from the plurality of sensors; and determining a plurality of regional sensor values for the plurality of sensor regions by, for each sensor region: identifying a region-specific model, the region-specific model specifying how the at least one regional sensor value for that sensor region is to be calculated; and generating the at least one regional sensor value for that sensor region by applying the region-specific model to the sensor readings obtained from the at least one region-associated sensor corresponding to that sensor region.

The method can include outputting the plurality of regional sensor values.

The method can include identifying one or more failed sensors in the plurality of sensors; and for each failed sensor, omitting the sensor readings corresponding to that failed sensor from the step of generating the regional sensor value for the corresponding sensor region.

The one or more failed sensors can be identified using a sensor-break algorithm.

The method can include applying the sensor-break algorithm on a continual basis.

The plurality of sensor regions can include at least one multi-sensor region, each multi-sensor region including a plurality of region-associated sensors.

For at least one of the multi-sensor regions the plurality of region-associated sensors can be defined to include a group of adjacent sensors.

The plurality of sensors can be electrically connected to an electronics module using a plurality of electrical connectors, the plurality of electrical connectors including a plurality of row connectors and a plurality of column connectors. Each sensor can be electrically connected to a pair of electrical connectors in the plurality of electrical connectors, the pair of electrical connectors connected to each sensor includes one row connector and one column connector, and the pair of electrical connectors connected to each sensor can be unique.

For each multi-sensor region the plurality of region-associated sensors can be connected to at least two different row connectors and at least two different column connectors.

For each multi-sensor region, each region-associated sensor can be electrically connected to a different row connector and column connector from every other region-associated sensor within that multi-sensor region.

The plurality of sensors can be grouped into the plurality of sensor regions based on a desired resolution, and the number of region-associated sensors within each sensor region can be determined at least in part based on the desired resolution.

At least two of the sensor regions can at least partially overlap.

The plurality of sensor regions can be predetermined in a preprocessing phase.

For each sensor region the region-specific model can be predetermined in the processing phase.

For at least one of the sensor regions the region-specific model can be defined as a single-value model configured to generate a single regional sensor value for the entire sensor region.

The single regional sensor value can be determined as one of a sensor value of the most activated region-associated sensor, an average sensor value of the at least one region-associated sensor, and a weighted-average sensor value of the at least one region-associated sensor.

The sensor value for a particular sensor can be adjusted based on a relative level of activation of one or more adjacent sensors.

For at least one of the sensor regions the region-specific model is defined as a multi-value model configured to generate a plurality of regional sensor values for the sensor region.

The multi-value model can include one of an interpolation model and an extrapolation model.

The carrier device can be a wearable device and the plurality of sensors can be provided by the wearable device.

The wearable device can be worn on a foot.

The wearable device can be an insole.

The plurality of sensors can be force sensors.

The first predetermined pattern can include at least 32 locations.

According to some aspects, there is also provided a system for determining regional sensor data. The system includes a plurality of sensors arranged in a first predetermined pattern, with each of the plurality of sensors arranged at respective locations on a carrier device, wherein the plurality of sensors are associated with a plurality of sensor regions, wherein each sensor region includes at least one region-associated sensor from the plurality of sensors; and one or more controllers communicatively coupled to the plurality of sensors, the one or more controllers configured to: obtain a corresponding plurality of sensor readings from the plurality of sensors; determine a plurality of regional sensor values for the plurality of sensor regions by, for each sensor region: identify a region-specific model, the region-specific model specifying how the at least one regional sensor value for that sensor region is to be calculated; and generate the at least one regional sensor value for that sensor region by applying the region-specific model to the sensor readings obtained from the at least one region-associated sensor corresponding to that sensor region.

The one or more controllers can be further configured to output the plurality of regional sensor values.

The one or more controllers can be further configured to: identify one or more failed sensors in the plurality of sensors; and for each failed sensor, omit the sensor readings corresponding to that failed sensor from the step of generating the regional sensor value for the corresponding sensor region.

The one or more controllers can be configured to identify the one or more failed sensors using a sensor-break algorithm.

The one or more controllers can be configured to apply the sensor-break algorithm on a continual basis.

The plurality of sensor regions can include at least one multi-sensor region, each multi-sensor region including a plurality of region-associated sensors.

For at least one of the multi-sensor regions the plurality of region-associated sensors can be defined to include a group of adjacent sensors.

The plurality of sensors can be electrically connected to an electronics module using a plurality of electrical connectors, the plurality of electrical connectors including a plurality of row connectors and a plurality of column connectors. Each sensor can be electrically connected to a pair of electrical connectors in the plurality of electrical connectors, the pair of electrical connectors connected to each sensor includes one row connector and one column connector, and the pair of electrical connectors connected to each sensor can be unique.

For each multi-sensor region the plurality of region-associated sensors can be connected to at least two different row connectors and at least two different column connectors.

For each multi-sensor region, each region-associated sensor can be electrically connected to a different row connector and column connector from every other region-associated sensor within that multi-sensor region.

The plurality of sensors can be grouped into the plurality of sensor regions based on a desired resolution, and the number of region-associated sensors within each sensor region can be determined at least in part based on the desired resolution.

At least two of the sensor regions can at least partially overlap.

The plurality of sensor regions can be predetermined in a preprocessing phase.

For each sensor region the region-specific model can be predetermined in the processing phase.

For at least one of the sensor regions the region-specific model can be defined as a single-value model configured to generate a single regional sensor value for the entire sensor region.

The single regional sensor value can be determined as one of a sensor value of the most activated region-associated sensor, an average sensor value of the at least one region-associated sensor, and a weighted-average sensor value of the at least one region-associated sensor.

The one or more controllers can be further configured to adjust the sensor value for a particular sensor based on a relative level of activation of one or more adjacent sensors.

For at least one of the sensor regions the region-specific model can be defined as a multi-value model configured to generate a plurality of regional sensor values for the sensor region.

The multi-value model can include one of an interpolation model and an extrapolation model.

The system can include the carrier device, the carrier device can be a wearable device, and the plurality of sensors can be provided by the wearable device.

The wearable device can be worn on a foot.

The wearable device can be an insole.

The plurality of sensors can be force sensors.

The first predetermined pattern can include at least 32 locations.

According to some aspects, there is provided a non-transitory computer readable medium storing computer-executable instructions, which, when executed by a computer processor, cause the computer processor to carry out a method for determining regional sensor data. The method includes: obtaining a plurality of sensor readings from a corresponding plurality of sensors, the plurality of sensors arranged in a first predetermined pattern, wherein the first predetermined pattern maps each of the plurality of sensors to respective locations on a carrier device and the plurality of sensors are grouped into a plurality of sensor regions, wherein each sensor region includes at least one region-associated sensor from the plurality of sensors; and determining a plurality of regional sensor values for the plurality of sensor regions by, for each sensor region: identifying a region-specific model, the region-specific model specifying how the at least one regional sensor value for that sensor region is to be calculated; and generating the at least one regional sensor value for that sensor region by applying the region-specific model to the sensor readings obtained from the at least one region-associated sensor corresponding to that sensor region.

The non-transitory computer readable medium can be configured to store computer-executable instructions, which, when executed by a computer processor, cause the computer processor to carry out a method for determining regional sensor data, where the method is described herein.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a block diagram illustrating an example of a system for determining regional sensor data;

FIG. 2A is a diagram illustrating an example of a sensing unit that can be used in the system of FIG. 1 showing a first set of connectors;

FIG. 2B is a diagram illustrating the example sensing unit shown in FIG. 2A with a second set of connectors;

FIG. 2C is a diagram illustrating the example sensing unit shown in FIG. 2A with the first set and second set of connectors;

FIG. 3 is a diagram illustrating a portion of the sensing unit of FIG. 2A with a conductivity failure;

FIG. 4 is a flowchart illustrating an example of a method for determining regional sensor data;

FIG. 5 is a flowchart illustrating an example of a preprocessing method that may be used with the method shown in FIG. 4;

FIG. 6A is a diagram illustrating a portion of the sensing unit of FIG. 2A showing the first set of connectors with the sensors grouped into example sensor regions;

FIG. 6B is a diagram illustrating a portion of the sensing unit of FIG. 2B showing the second set of connectors with the sensors grouped into the example sensor regions shown in FIG. 6A;

FIG. 7A is a diagram illustrating the sensing unit of FIG. 2A showing the first set of connectors with the sensors grouped into another example of sensor regions;

FIG. 7B is a diagram illustrating the sensing unit of FIG. 2B showing the second set of connectors with the sensors grouped into the example sensor regions shown in FIG. 7A;

FIG. 8A is a diagram illustrating the sensing unit of FIG. 2A showing the first set of connectors with the sensors grouped into another example of sensor regions;

FIG. 8B is a diagram illustrating the sensing unit of FIG. 2B showing the second set of connectors with the sensors grouped into the example sensor regions shown in FIG. 8A;

FIG. 9A is a diagram illustrating the sensing unit of FIG. 2A showing the first set of connectors with the sensors grouped into another example of sensor regions;

FIG. 9B is a diagram illustrating the sensing unit of FIG. 2B showing the second set of connectors with the sensors grouped into the example sensor regions shown in FIG. 9A;

FIG. 10A is a diagram illustrating the sensing unit of FIG. 2C showing the first and second set of connectors with the sensors grouped into the example sensor regions shown in FIG. 8A and example sensor values;

FIG. 10B is a diagram illustrating an example of regional sensor values generated from the example sensor values shown in FIG. 10A;

FIG. 10C is a diagram illustrating the sensing unit of FIG. 2A showing the first set of connectors with failed connector sections corresponding to the example sensor values shown in FIG. 10A;

FIG. 11A is a diagram illustrating the sensing unit of FIG. 2A showing the first set of connectors with example sensor values corresponding to the example sensor values shown in FIG. 10A;

FIG. 11B is a diagram illustrating another example of regional sensor values generated from the example sensor values shown in FIG. 11A;

FIG. 12A is a diagram illustrating another example of a sensing unit that can be used in the system of FIG. 1 showing an alternative first set of connectors;

FIG. 12B is a diagram illustrating the sensing unit of FIG. 12A showing an alternative second set of connectors;

FIG. 12C is a diagram illustrating the sensing unit of FIG. 12A showing the alternative first and second sets of connectors; and

FIG. 13 is a diagram illustrating a screenshot of a dashboard that may be output by the system of FIG. 1.

### DETAILED DESCRIPTION

Various apparatuses or processes or compositions will be described below to provide an example of an embodiment of the claimed subject matter. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements. In addition, numerous specific details are set forth in order to provide a thorough understanding of the subject matter described herein. However, it will be understood by those of ordinary skill in the art that the subject matter described herein may be practiced without these specific details. In other instances, well-known methods, procedures and components have not been described in detail so as not to obscure the subject matter described herein. The description is not to be considered as limiting the scope of the subject matter described herein.

The terms “coupled” or “coupling” as used herein can have several different meanings depending in the context in which these terms are used. For example, the terms coupled or coupling can have a mechanical, electrical or communicative connotation. For example, as used herein, the terms coupled or coupling can indicate that two elements or devices can be directly connected to one another or connected to one another through one or more intermediate elements or devices via an electrical element, electrical signal, or a mechanical element depending on the particular context. Furthermore, the term “communicative coupling” may be used to indicate that an element or device can electrically, optically, or wirelessly send data to another element or device as well as receive data from another element or device.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

Terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

Described herein are systems, methods and devices for collecting sensor data and determining regional sensor values for a plurality of sensors mapped to a carrier unit. The systems, methods, and devices can use sensors attached to, or contained within, a carrier unit such as a wearable device or fitness equipment to measure and monitor data relating to movement or activity of an individual.

The sensors can be force sensors and can be provided in the insole of a shoe or within the footwear worn by the individual. As used herein, the term “force” is used broadly and can refer to raw force (i.e. with units of N), or pressure resulting from a raw force (i.e. with units of N/m²). The force data acquired by the force sensors can be used to determine the level of force applied by an individual's foot when performing various activities such as walking, running, or jumping for example. This force data can be used to derive additional force derivatives or force-based metrics, such as the force output or the center of pressure for the individual. The force data, and other data derived therefrom, can be used for tracking and monitoring various parameters that may be useful for medical, fitness, athletic, entertainment or other purposes.

The systems, methods, and devices described herein may be implemented as a combination of hardware or software. In some cases, the systems, methods, and devices described herein may be implemented, at least in part, by using one or more computer programs, executing on one or more programmable devices including at least one processing element, and a data storage element (including volatile and non-volatile memory and/or storage elements). These devices may also have at least one input device (e.g. a pushbutton keyboard, mouse, a touchscreen, and the like), and at least one output device (e.g. a display screen, a printer, a wireless radio, and the like) depending on the nature of the device.

Some elements that are used to implement at least part of the systems, methods, and devices described herein may be implemented via software that is written in a high-level procedural language such as object-oriented programming. Accordingly, the program code may be written in any suitable programming language such as Python or C for example. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, magnetic disk, optical disc) or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods described herein may be capable of being distributed in a computer program product including a computer readable medium that bears computer usable instructions for one or more processors. The medium may be provided in various forms, including non-transitory forms such as, but not limited to, one or more diskettes, compact disks, tapes, chips, and magnetic and electronic storage. Alternatively, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

The present disclosure relates to a system, method, and computer program product that can be used to determine regional sensor values based on sensor readings from a plurality of sensors. The plurality of sensors can be positioned in a predetermined arrangement or pattern that is mapped to respective locations on a carrier device such as a wearable device or fitness equipment. A plurality of sensor readings can be received from the plurality of sensors.

The plurality of sensors can be grouped into a plurality of sensor regions. Regional sensor values can be determined for each sensor region based on the sensor readings from the sensors in the respective region. In some examples, the sensor regions can be defined to ensure that regional sensor values can be determined even when one or more sensors within a region fails and/or loses connectivity due to a failure of a signal connector. Alternatively, or in addition, the sensor regions can be defined based on a desired measurement resolution for the plurality of sensors and/or a particular use of the sensors (e.g. gaming applications, injury monitoring, etc.).

Each sensor region can be associated with a corresponding region-specific model. Each region-specific model can define how the regional sensor values are determined for a given sensor region based on the sensor readings from the individual sensors within that region. The sensor readings from the sensors within a given region can be provided as inputs to the corresponding region-specific model in order to determine the regional sensor value(s) for that region.

The plurality of sensor readings and the plurality of regional sensor values can then be output. The output data can be used for various purposes, such as providing an individual with feedback on the sensor readings, as inputs to a gaming application (e.g. to control the movement of an avatar within a virtual environment) and/or for further analysis to determine derived sensor data.

In general, the sensors can be connected to signal connectors that carry signals to and from the sensors. For example, the connectors may be electrical connectors that carry electricity to and from the sensors to allow the sensor readings to be determined. The connectors can be provided as signal traces such as conductive ink traces that are printed or etched onto a substrate. The connectors can carry current to and from sensors.

The present disclosure can be applied to a plurality of sensors that are arranged in a matrix-based sensor array or a non-matrix-based sensor array. In a matrix-based array, each sensor is connected to a pair of connectors namely a row connector and a column connector. Each sensor in the matrix-based array is associated with a unique row connector and column connector pair (also referred to as a unique row and column coordinate combination).

In order for the sensor readings to be output, the signals are carried by the connectors between the sensors and an electronics module associated with the sensing unit. If there are failures in the connectors used to carry signals between the sensors and the electronics module (referred to as conductivity failures), the sensing unit may be rendered unusable. For example, a failed sensor or a sensor that is no longer connected to the electronics module due to a conductivity failure may appear as a “blackout” (e.g. a null value) in a mapping of the sensor values. This can be disruptive for users and can also reduce the accuracy of metrics derived from the sensor readings due to the loss of sensor information. Hardware modifications to improve sensor durability or repairs to failed sensor can be expensive and may be impractical depending on the sensing unit. In a matrix-based array each row connector and each column connector is typically connected to multiple sensors. As a result, a conductivity failure in one connector can result in an inability to determine the sensor readings from multiple sensors.

In some examples, the present disclosure can be applied to address conductivity failures without requiring modifications or repairs to the sensing unit. This can serve to elongate the usable life of the sensing unit and improve the overall user experience.

Depending on the desired use for a sensing unit, the desired level of measurement resolution (also referred to as measurement granularity) can vary. For example, the present disclosure can be used in the context of a wearable device worn on a user's foot (e.g. a sensorized insole) that includes a plurality of force sensors. In certain applications, such as sports performance applications, a moderate or high degree of measurement resolution is required. In such applications, it is critical that the data collected from the sensors be abundant and accurate, in order to provide athletes with usable insights into their biomechanics. However, other applications such as gaming often do not require the same level of resolution. In fact, the increased resolution may be excessive given that it can be computationally expensive to analyze data from more sensors than required.

Accordingly, in some examples of the present disclosure the sensors within a sensing unit can be grouped into sensor regions based at least in part on the desired resolution of a particular application. The sensor regions and associated region-specific models can be selected to provide regional sensor values that correspond to the desired resolution. This can allow the same sensing unit to be used effectively in applications requiring different levels of measurement resolution.

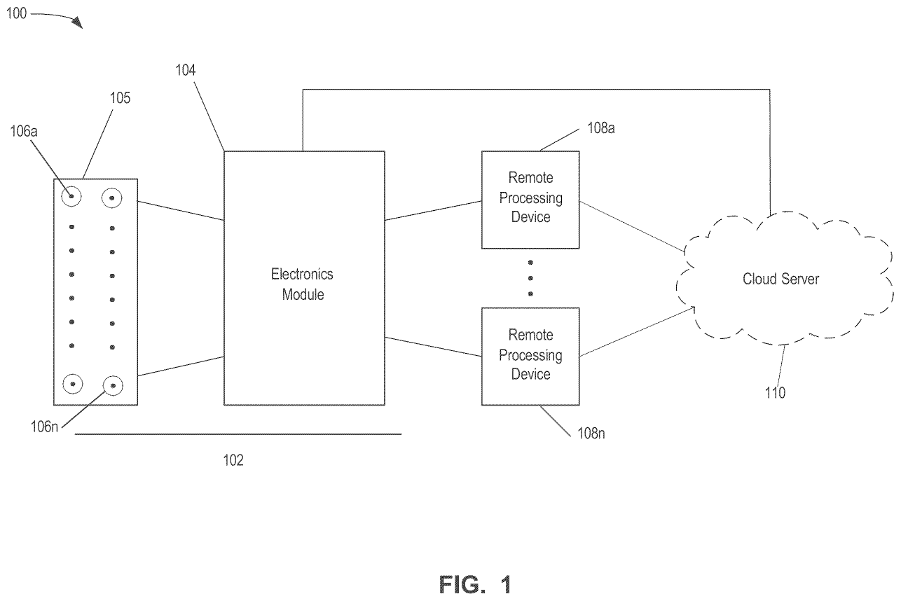

Referring now to FIG. 1, shown therein is a block diagram illustrating an example system 100 that can be used to collect sensor data for a user. System 100 can also be used to determine regional sensor values for the user using the collected sensor data.

System 100 includes an input unit 102 (also referred to herein as an input device), one or more processing devices 108 (also referred to herein as a receiving device or an output device) and optionally a remote cloud server 110. As will be described in further detail below, the input unit 102 may for example be combined with, or integrated into, a carrier unit such as a wearable device or a piece of fitness equipment.

Input unit 102 generally includes a sensing unit 105. The sensing unit 105 can include a plurality of sensors 106a-106n. The plurality of sensors 106a-106n can be arranged in a first predetermined pattern that maps each sensor 106 to a corresponding location of the carrier unit.

The carrier unit can be configured to hold the sensors 106 in contact with (or close proximity to) an individual's body to allow the sensors 106 to measure an aspect of the activity being performed by the individual. The plurality of sensors 106a-106n may be configured to measure a particular sensed variable at a location of an individual's body when the carrier unit is engaged with the individual's body (e.g. when the individual is wearing a wearable device containing the sensors 106 or when the individual is using fitness equipment containing the sensors 106).

In some examples, the carrier unit may include one or more wearable devices. The wearable devices can be manufactured of various materials such as fabric, cloth, polymer, or foam materials suitable for being worn close to, or in contact with, a user's skin. All or a portion of the wearable device may be made of breathable materials to increase comfort while a user is performing an activity.

In some examples, the wearable device may be formed into a garment or form of apparel such as a band, headwear, a shirt, shorts, a sock, a shoe, a sleeve, and a glove (e.g. a tactile glove). Some wearable devices such as socks or sleeves may be in direct contact with a user's skin. Some wearable devices, such as shoes, may not be in direct contact with a user's skin but still positioned within sufficient proximity to a user's body to allow the sensors to acquire the desired readings.

In some cases, the wearable device may be a compression-fit garment. The compression-fit garment may be manufactured from a material that is compressive. A compression-fit garment may minimize the impact from “motion artifacts” by reducing the relative movement of the wearable device with respect to a target location on the individual's body. In some cases, the wearable device may also include anti-slip components on the skin-facing surface. For example, a silicone grip may be provided on the skin-facing surface of the wearable device to further reduce the potential for motion artifacts.

In some examples, the wearable device may be worn on a foot. For example, the wearable device may be a shoe, a sock, an insole or a portion of a shoe, a sock, or an insole. The wearable device may include a deformable material, such as foam. This may be particularly useful where the wearable device is worn underfoot, as in a shoe or insole.

The plurality of sensors 106a-106n can be positioned to acquire sensor reading from specified locations on an individual's body (via the arrangement of the sensors on the carrier unit). The sensors 106 can be integrated into the material of the carrier unit (e.g. integrated into a wearable device or fitness equipment). Alternatively, the sensors 106 can be affixed or attached to the carrier unit, e.g. printed, glued, laminated or ironed onto a surface, or between layers, of a wearable device or fitness equipment.

In some examples, the carrier unit may include fitness equipment. The fitness equipment may include various types of fitness equipment on which a user can exert force while performing an activity. For example, the carrier unit may be fitness equipment such as an exercise mat, a fitness bench, a bar (e.g. a squat rack or a pull-up bar), a treadmill, or a bicycle seat for a bicycle or stationary bicycle.

For clarity, the below description relates to a carrier unit in the form of an insole. The insole carrier unit may be provided in various forms, such as an insert for footwear or integrated into a shoe. However, other carrier units may be implemented using the systems and methods described herein, such as the wearable devices and fitness equipment described above. In addition, the systems and methods described herein can also be implemented with sensor arrays used for applications other than monitoring human movement and activity, such as temperature sensor arrays used with solar panels or other more general sensor array applications.

The below description relates to an insole in which the plurality of sensors 106 are force sensors. However, alternative types of sensors may be used such as, for example, optical sensors, temperature sensors, or electromagnetic sensors.

In addition, various types of force sensors may be used, such as force sensing resistors (also referred to as “sensels” or sensing elements), pressure sensors, piezoelectric tactile sensors, elasto-resistive sensors, capacitive sensors or more generally any type of force sensor that can be integrated into a carrier device such as a wearable device or fitness equipment for example.

As shown in FIG. 1, input unit 102 includes an electronics module 104 coupled to the plurality of sensors 106. In some cases, the electronics module 104 can include a power supply, a processor, a memory, a signal acquisition unit operatively coupled to the processor and to the plurality of sensors 106, and a wireless communication module operatively coupled to the processor.

Generally, the sensing unit refers to the plurality of sensors 106 and the signal acquisition unit. The signal acquisition unit may provide initial analog processing of signals acquired using the sensors 106, such as amplification. The signal acquisition unit may also include an analog-to-digital converter to convert the acquired signals from the continuous time domain to a discrete time domain. The analog-to-digital converter may then provide the digitized data to the processor for further analysis or for communication to a remote processing device 108 or remote cloud server 110 for further analysis.

Optionally, the electronics module 104 may include a processor configured to perform the signal processing and analysis. In such cases, the processor on the electronics module may be configured to process the received sensor readings in order to determine regional sensor values. In some cases, the processor may be coupled to the communication module (and thereby the sensing unit) using a wired connection such as Universal Serial Bus (USB) or other port.

The electronics module 104 can be communicatively coupled to one or more remote processing devices 108a-108n, e.g. using a wireless communication module (e.g., Bluetooth, Bluetooth Low-Energy, WiFi, ANT+ IEEE 802.11, etc.). The remote processing devices 108 can be any type of processing device such as a personal computer, a tablet, and a mobile device such as a smartphone, a smartwatch or a wristband for example. The electronics modules 104 can also be communicatively coupled to a remote cloud server 110 over, for example, a wide area network such as the Internet.

Each remote processing device 108 and optional remote cloud server 110 typically includes a processing unit, an output device (such as a display, speaker, or tactile feedback device), a user interface, an interface unit for communicating with other devices, Input/Output (I/O) hardware, a wireless unit (e.g. a radio that communicates using CDMA, GSM, GPRS or Bluetooth protocol according to standards such as IEEE 802.11a, 802.11b, 802.11g, or 802.11n), a power unit and a memory unit. The memory unit can include RAM, ROM, one or more hard drives, one or more flash drives or some other suitable data storage elements such as disk drives, etc.

The processing unit controls the operation of the remote processing device 108 or the remote cloud server 110 and can be any suitable processor, controller or digital signal processor that can provide sufficient processing power depending on the desired configuration, purposes and requirements of the system 100.

The display can be any suitable display that provides visual information. For instance, the display can be a cathode ray tube, a flat-screen monitor and the like if the remote processing device 108 or remote cloud server 110 is a desktop computer. In other cases, the display can be a display suitable for a laptop, tablet or handheld device such as an LCD-based display and the like.

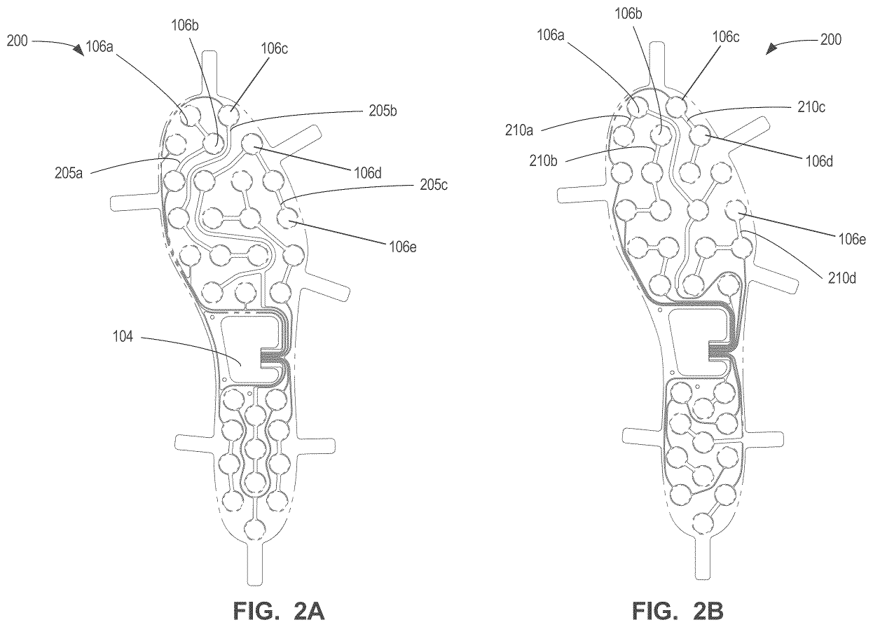

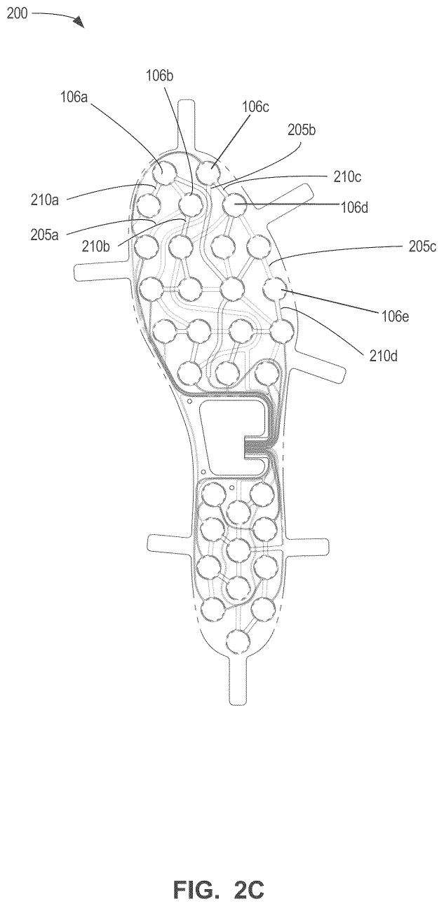

The plurality of sensors 106 may be arranged into a sensor array. As used herein, the term sensor array refers to a series of sensors arranged in a defined grid. The plurality of sensors 106 can be arranged in various types of sensor arrays. For example, the plurality of sensors 106 can be provided as a set of discrete sensors (see e.g. FIGS. 2A-2C). A discrete sensor is an individual sensor that acquires a sensor reading at a single location. A set of discrete sensors generally refers to multiple discrete sensors that are arranged in a spaced apart relationship in a sensing unit.

Sensors 106a-106n may be arranged in a sparse array of discrete sensors that includes void locations where no sensors 106 are located. Alternatively, sensors 106a-106n may be arranged in a continuous or dense sensor array in which sensors 106 are arranged in a continuous, or substantially continuous manner, across the grid.

Discrete sensors can provide an inexpensive alternative to dense sensor arrays for many applications. However, because no sensors are positioned in the interstitial locations between the discrete sensors and the void locations external to the set of discrete sensors, no actual sensor readings can be acquired for these locations. Accordingly, depending on the desired resolution for the force sensor data, sensor readings may be estimated (rather than measured) at the interstitial locations and at the void locations external to the set of discrete sensors in order to provide sensor data with similar resolution to a dense sensor array. Alternatively, where lower resolution force sensor data is sufficient, sensor readings may not necessarily be estimated.

Various interpolation and extrapolation techniques may be used to estimate sensor values at interstitial locations and external void locations. The plurality of sensors 106 can also be connected to the electronics module 104 using signal connectors. In cases of sensor failure or conductivity failure, various interpolation and extrapolation techniques can be used to estimate sensor values at the locations of failed sensors or sensors that are not accessible due to a conductivity failure.

In some cases, sensor values may be estimated using the methods for synthesizing sensor data described in Applicant's co-pending patent application Ser. No. 17/988,468 filed on Nov. 16, 2022 entitled “SYSTEM AND METHOD FOR SYNTHESIZING SENSOR READINGS”, the entirety of which is incorporated herein by reference. In some cases, sensor values may be estimated using the methods for synthesizing sensor data described in Applicant's co-pending patent application Ser. No. 18/183,642 filed on Mar. 14, 2023 entitled “SYSTEM AND METHOD FOR DETERMINING USER-SPECIFIC ESTIMATION WEIGHTS FOR SYNTHESIZING SENSOR READINGS”, the entirety of which is incorporated herein by reference. In some examples, these methods can also be applied to estimate sensor values at the locations of failed sensors or sensors that are not accessible due to a conductivity failure.

System 100 can be configured to implement a method of determining regional sensor data that can overcome sensor failures and/or conductivity failures and/or allow for sensor values to be obtained effectively for different applications, including adjusting the measurement resolution of the sensing unit 102. The method of determining regional sensor data may be implemented using a controller of the input device 102, a remote processing device 108, or cloud server 110.

In some cases, system 100 may also track additional parameters or derived data from the sensor readings and regional sensor values. The sensor readings, regional sensor values, and derived data may be monitored, stored, and analyzed for the user. Aspects of the monitoring, storage and analysis of sensor readings and regional sensor values may be performed by one or more of the input unit 102, and/or a remote processing device 108, and/or the cloud server 110.

A remote cloud server 110 may provide additional processing resources not available on the input unit 102 or the remote processing device 108. For example, some aspects of processing the sensor readings acquired by the sensors 106 may be delegated to the cloud server 110 to conserve power resources on the input unit 102 or remote processing device 108. In some cases, the cloud server 100, input unit 102 and remote processing device 108 may communicate in real-time to provide timely feedback to a user regarding the sensor readings, regional sensor value and data derived therefrom and/or to use the sensor readings and regional sensor value for real-time applications such as gaming.

Referring now to FIGS. 2A-2C, shown therein is an example of a sensing unit 200 that may be used with an insole. The insole is an example of an input device 102 that may be used in the system 100 shown in FIG. 1. The insole may be used to provide a plurality of force sensors for a footwear insert, such as the insert described in PCT Application No. PCT/CA2020/051520, the entirety of which is incorporated herein by reference.

As shown in FIG. 2A sensor unit 200 includes a plurality of sensors 106 and an electronics modules 104. The plurality of sensors 106 are coupled to the electronics module 104 using a plurality of connectors 205/210 (see FIG. 20). The connectors 205/210 provide a coupling interface between the plurality of sensors 106 and the electronics module 104. The coupling interface can allow signals from the sensors 106 (e.g. sensor readings) to be transmitted to the electronics module. In some cases, the coupling interface may also provide control or sampling signals from the electronics module to the sensors 106.

In some examples, connectors 205/210 can be provided as trace layers on the sensor unit 200. For example, the trace layers may be prepared from a low-resistance material such as copper, silver, gold, conductive ink, temperature resistive ink, or a combination of the above low-resistance materials. The trace layers may be printed, deposited, or etched onto a substrate of the sensor unit 200. Alternatively, the connectors 205/210 can be provided using other signal connectors such as electrical wires for example and may include additional coupling materials such as a solder. ACF bonds and/or conductive epoxies. The connection between the sensors 106 and the electronics module 104 can also include additional connectors components, including electronic connectors such as flat flex cable (FFC) connectors, flexible printed circuit (FCP) connectors, pogo pin connectors etc.

As illustrated, the plurality of sensors 106 are arranged in a predetermined pattern (also referred to as a sensor layout or predetermined sensor layout) where the sensors 106 are spaced apart from one another. The sensors 106 provide a set of discrete sensors that are distributed across the sensor unit 200.

In this layout, there are void locations where no actual sensor readings can be acquired. The void locations can include interstitial locations between the sensors 106 where no actual sensor readings can be acquired. The void locations can also include external void locations outside of the sensors 106 where no actual sensor readings can be acquired.

The predetermined pattern of sensors 106 can include at least 32 locations. As illustrated in the example of FIGS. 2A-2C, the predetermined pattern of sensors 106 in sensor unit 202 includes exactly 32 locations.

As shown in the example of FIGS. 2A-2C, the sensors 106 are coupled to the electronics module 104 using a matrix-based arrangement of connectors 205 and 210. The plurality of connectors includes a plurality of row connectors 205 (see e.g. FIGS. 2A and 2C) and a plurality of column connectors (see e.g. FIGS. 2B and 2C). Each sensor 106 is connected to a pair of connectors and the pair of connectors connected to each sensor includes one row connector 205 and one column connector 210. As shown in FIG. 2C, each sensor 106 is connected to a different row connector 205 and column connector 210 combination (a unique pair) (also referred to as each sensor 106 being associated with a unique row and column coordinate combination).

FIG. 2A shows the sensing unit 200 with the row connectors 205 displayed and the column connectors 210 omitted. As shown in FIG. 2A, sensors 106a and 106b are both connected to row connector 205a. Sensor 106c is connected to a different row connector 205b. Sensors 106d and 106e are also connected to a shared row connector 205c that is different from the row connectors connected to sensors 106a-106c.

FIG. 2B shows the sensing unit 200 with the column connectors 210 displayed and the row connectors 205 omitted. As shown in FIG. 2B, sensor 106a is connected to column connector 210a but sensor 106b is connected to a different column connector 210b. Sensor 106c is connected to a different column connector 210c and sensor 106d is also connected to column connector 210c. Sensor 106e is connected to a column connector 210d that is different from the column connectors connected to sensors 106a-106d.

FIG. 2C shows the sensing unit 200 with both the row connectors 205 and the column connectors 210 displayed. As shown in FIG. 2C, each sensor 106a-106e is connected to a different row connector and column connector pair.

As noted above, the sensors 106 are coupled to the electronics module 104 using a matrix-based arrangement of connectors 205 and 210. FIGS. 2A-2C illustrate a first example of a matrix-based layout of row connectors 205 and column connectors 210. However, alternative matrix-based connector layouts may also be used.

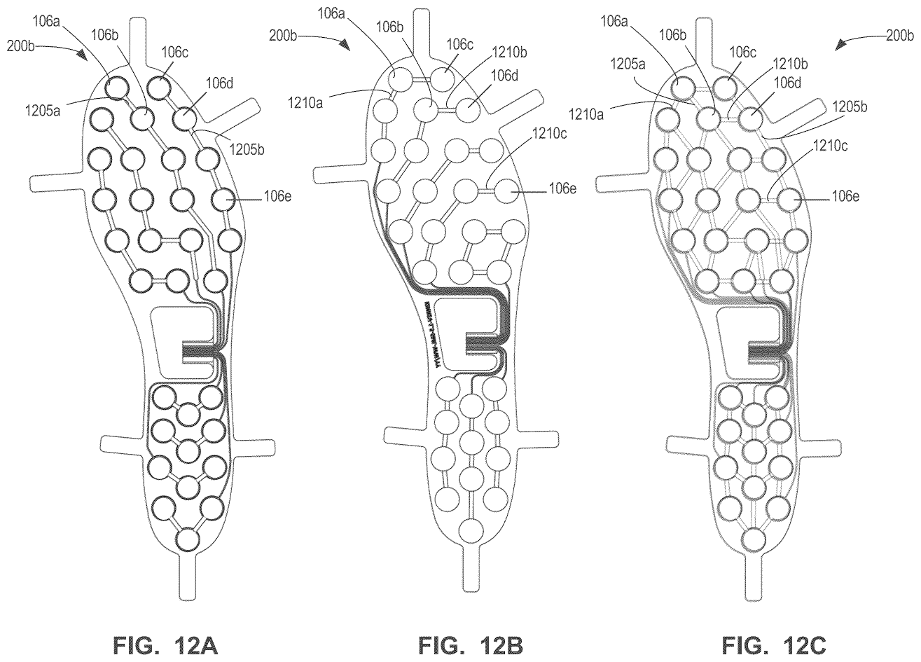

FIGS. 12A-12C illustrate an alternative example of a sensing unit 200b. Sensing unit 200b is generally similar to sensing unit 200 except that the row connectors 1205 and column connectors 1210 in sensing unit 200b have a different arrangement as compared to the row connectors 205 and column connectors 210 in sensing unit 200. Similar to sensing unit 200, in sensing unit 200b each sensor 106 is connected to a different row connector 1205 and column connector 1210 combination (a unique pair) (see FIG. 12C).

FIG. 12A shows the sensing unit 200b with the row connectors 1205 displayed and the column connectors 1210 omitted. As shown in FIG. 12A, sensors 106a and 106b are both connected to row connector 1205a. Sensors 106c, 106d and 106e are all connected to a second row connector 1205b.

FIG. 12B shows the sensing unit 200b with the column connectors 1210 displayed and the row connectors 1205 omitted. As shown in FIG. 12B, sensors 106a and 106c are connected to column connector 1210a but sensor 106b is connected to a different column connector 1210b, and sensor 106d is also connected to column connector 1210b. Sensor 106e is connected to a column connector 1210c that is different from the column connectors connected to sensors 106a-106d.

FIG. 12C shows the sensing unit 200b with both the row connectors 1205 and the column connectors 1210 displayed. As shown in FIG. 12C, each sensor 106a-106e is connected to a different row connector and column connector pair.

Matrix-based sensor arrays are susceptible to conductivity failures in the connectors 205/210 used to couple signals between the sensors 106 and the electronics module 104. Conductivity failures can occur in various ways. For example, conductivity failures can occur when the traces connecting the sensors on the sensor array, the traces on the printed circuit board (PCB), or the bonding materials that connect the sensor array to the PCB crack or separate, which causes a loss of conductivity to the downstream sensors. The sensors 106 themselves can also fail, although this is a rare occurrence.

In addition, sensing units with non-matrix-based connector layouts (e.g. layouts where each sensor is connected to the electronics module using a detected connector) may also be used. In layouts where each sensor is uniquely connected to the electronics module (i.e. a non-matrix-based sensor array), a failed connector would only result in the failure of a single sensor. However, such configurations are often more costly and can be problematic for space-sensitive applications since additional connectors are typically required to enable this connectivity. In any event, conductivity failures and sensor failures still occur with non-matrix-based connector layouts although the impact may be lessened in some cases.

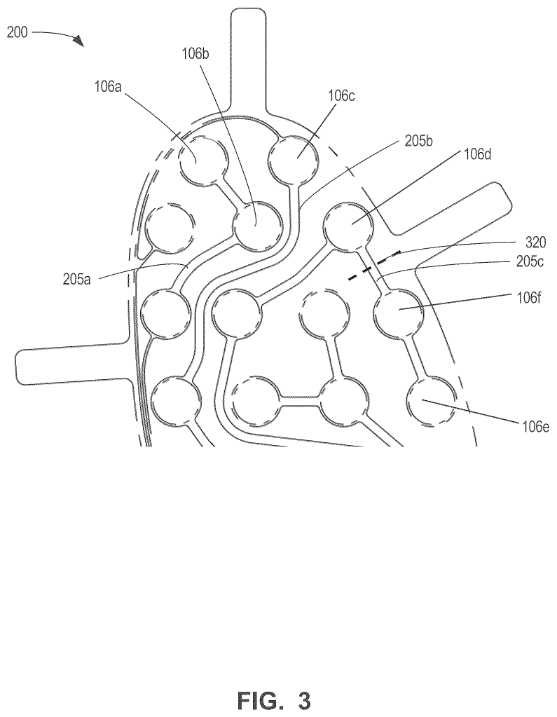

FIG. 3 shows an example of a conductivity failure in a portion of the sensing unit 200. As shown in FIG. 3, a conductivity failure 320 occurred at the midpoint of row connector 205c (e.g. a broken trace line). Due to the conductivity failure, the sensors 106e and 106f downstream from the conductivity failure 320 have lost connectivity to the electronics module. As a result, sensor readings are not retrievable from sensors 106e and 106f. Although sensor 106d is also connected to connector 205c, it has not lost connectivity to the electronics module since it is upstream of the conductivity failure 320. The systems and methods described herein may allow sensor values to be determined even in cases where conductivity failures have occurred.

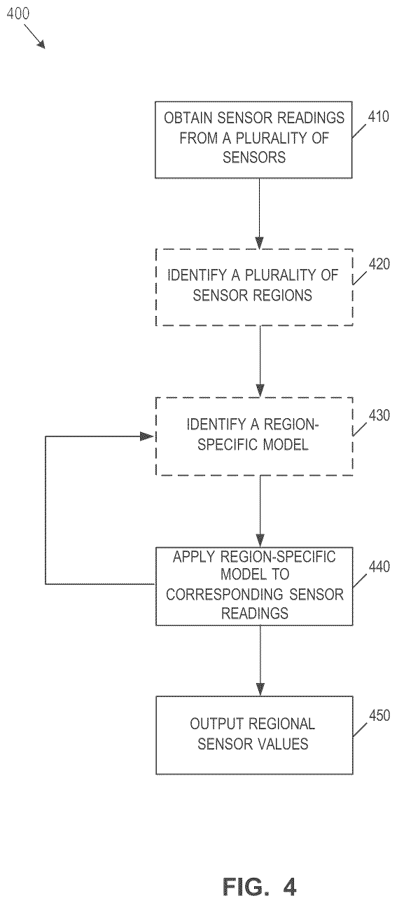

Referring now to FIG. 4, shown therein is an example method 400 for determining regional sensor data. The method 400 may be used to determine regional sensor data for a carrier unit such as a wearable device or fitness equipment. The method 400 may be used with a plurality of sensors configured to measure human movement or human activity, such as sensors 106 for example. Method 400 is an example of a method for determining regional sensor data in which sensors within a sensor array are grouped into a plurality of sensor regions.

At 410, a plurality of sensor readings can be obtained from a corresponding plurality of sensors (e.g. sensors 106). The sensor readings can be obtained from the sensors on an ongoing basis (e.g. sensor readings can be collected continuously while the system 100 is in use).

The sensors can be positioned at specified locations on a carrier unit such as a wearable device or a piece of equipment. The plurality of sensors can be arranged in a first predetermined pattern, such as the pattern shown in FIG. 2A. The first predetermined pattern can map each of the plurality of sensors to respective locations on the carrier unit. Accordingly, when the carrier unit is engaged with an individual's body, the sensors can be mapped to respective locations on the individual's body.

The first predetermined pattern can include different numbers of sensor locations depending on the type of carrier unit and/or the type of sensor. For example, the first predetermined pattern can include at least 32 locations. At least 32 sensor locations may be particularly desirable where the sensor unit is arranged to acquire data from under an individual's foot in order to allow regional sensor values to be determined with a sufficient degree of resolution for various applications.

The sensors can be configured to measure data relating to human activity. As shown in FIGS. 2A-2C, the plurality of sensors may be force sensors mapped to specific locations of an insole. The force sensors can measure force applied to the insole during physical activities, such as walking, running, jumping, or cycling for example.

Optionally, method 400 can include a step of identifying one or more failed sensors in the plurality of sensors. This may occur prior to step 410 in some examples. For each failed sensor identified, the method can include omitting the sensor readings corresponding to that failed sensor from the step of generating the regional sensor value for the corresponding sensor region.

Failed sensors may refer to sensors from which sensor readings are not obtainable or where the sensor readings are known or expected to be faulty. For example, failed sensors can refer to sensors that have failed as well as sensors that have lost connectivity to an electronics module due to intervening conductivity failures. Optionally, sensors that are expected to fail or are suspected to have failed (but have not been confirmed to have failed) can also be treated as if they have failed and the corresponding sensor readings can be omitted.

Failed sensors can be identified in various ways. For example, failed sensors can be identified by automated or manual analysis of the sensor readings obtained from the plurality of sensors.

A force sensor that is affected by a conductivity failure typically produces sensor readings of 0 when force/pressure is applied to the sensor. If a sensor is nearing failure, the sensor reading values may intermittently drop to 0 or near to 0. These intermittent drops in signal values can indicate a failure or imminent failure of the sensor.

In some cases, a force sensor that is affected by a conductivity failure may output a sensor reading that achieves 100% of the sensor's full scale range despite the absence of an applied load. Sensor readings at or near the maximum achievable sensor value despite the absence of any load can indicate a failure or imminent failure of the sensor.

In some examples, a sensor break algorithm can be applied to identify the failed sensors. The sensor break algorithm can be defined to identify failed sensors as sensors producing sensor readings of 0 when force/pressure is applied to the sensor and/or sensors where the sensor readings intermittently drop to 0 or near to 0. The sensor break algorithm can also be defined to identify failed sensors as sensors that produce maximum readings when no force/pressure is applied.

Furthermore, if a failed sensor has been identified along the same connector, the sensor-break algorithm can be defined to identify additional sensors coupled to the same connector that are likely to fail.

Optionally, the process of identifying failed sensors can be repeated over time (e.g. on a continual basis). For example, the process of identifying failed sensors can be executed each time the sensor array is used, the algorithm can be executed after every hour of use, etc. For example, a sensor-break algorithm can be automatically repeated on a regular basis at various frequencies and/or based on use.

At 420, a plurality of sensor regions associated with the sensor readings can be identified. The plurality of sensors in the sensing unit can be grouped into a plurality of sensor regions. Each sensor region can include at least one region-associated sensor from the plurality of sensors.

The sensors can be grouped into the sensor regions in various ways. In some examples, the sensors can be grouped into sensor regions during a pre-processing phase. Alternatively, or in addition, the sensors can be grouped into sensor regions during real-time operation of the sensing unit.

Optionally, the grouping of sensors into sensor regions can be modified or adjusted during operation of the sensing unit. For example, the sensor regions may be modified in response to a change in the activity being performed by a user (e.g. in order to adjust the measurement resolution for the activity being performed). The sensor regions may also be modified in response to determining that the operational status of one or more sensors has changed due to a sensor failure or conductivity failure (e.g. to optimize the sensor regions to account for the newly failed sensor(s)).

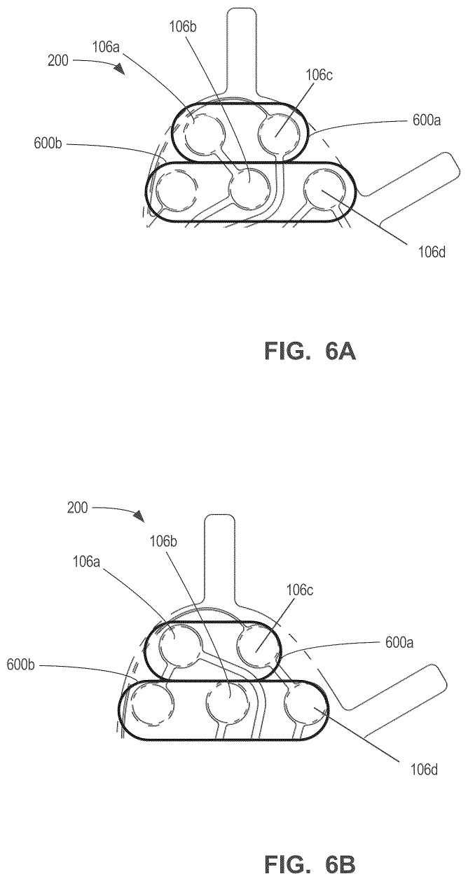

FIGS. 6A and 6B illustrate an example arrangement of sensor regions 600a and 600b for the forefoot region of the sensing unit 200 shown in FIGS. 2A-2C. FIG. 6A shows the forefoot region of the sensing unit 200 with the row traces 205 shown and the column traces 210 omitted. FIG. 6B shows the forefoot region of the sensing unit 200 with the column traces 210 shown and the row traces 205 omitted.

As shown in FIGS. 6A and 6B, sensors 106a and 106c are grouped into a first sensor region 600a while sensors 106b and 106d are grouped into a second sensor region 600b. The arrangement of sensor regions 600a and 600b shown in FIGS. 6A-6B is an example sensor region grouping in which each sensor 106 in a given region has a different row connector and a different column connector. Each of the sensor regions 600 shown in FIG. 6A and FIG. 6B is a multi-sensor region that includes a plurality of region-associated sensors.

As can be seen in Table 1 below, the number of row and column connectors in the sensor regions is the same as the number of sensors in the regions. This indicates that row and column connectors are not repeated within a region 600.

TABLE 1
Arrangement of Sensors Shown in FIGS. 6A and 6B

| | # OF | # OF ROW | # OF COLUMN |
|---|---|---|---|
| | SENSORS | CONNECTORS | CONNECTORS |
| REGION 600a | 2 | 2 | 2 |
| REGION 600b | 3 | 3 | 3 |

In the example shown in FIGS. 6A and 6B, each sensor 106 contained within a given region 600 is coupled to a different row connector and column connector than all other sensors contained in the region. This can ensure that in the event of a conductivity failure resulting in the failure of all sensors downstream from the conductivity failure, only a single region-associated sensor in each sensor region would fail.

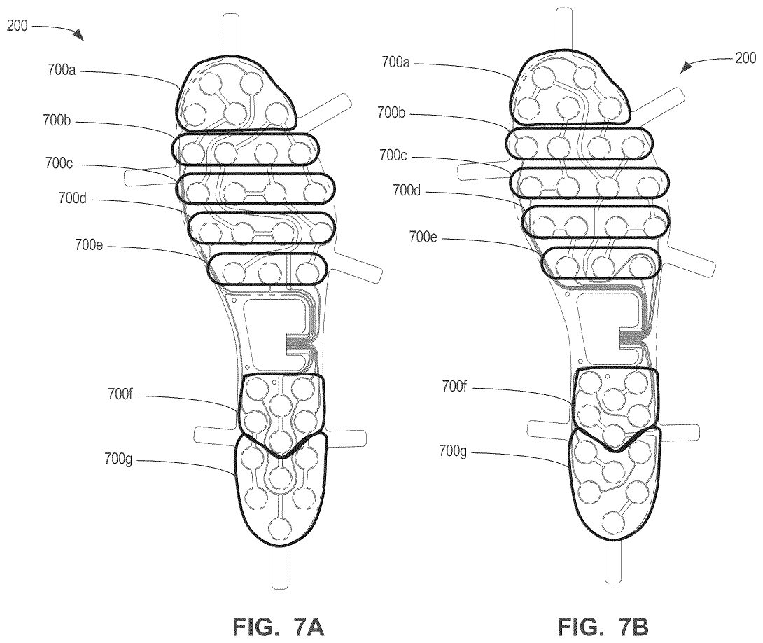

FIGS. 7A and 7B illustrate an example arrangement of sensor regions 700a-700g for the sensing unit 200 shown in FIGS. 2A-2C. FIG. 7A shows the sensing unit 200 with the row traces 205 shown and the column traces 210 omitted. FIG. 7B shows the sensing unit 200 with the column traces 210 shown and the row traces 205 omitted.

As shown in FIGS. 7A and 7B, each of the sensor regions 700 is a multi-sensor region that includes a plurality of region-associated sensors. The sensor regions 700 shown in FIGS. 7A and 7B are examples of sensor regions that are defined to include a group of adjacent sensors. Grouping adjacent sensor can be desirable since the values from functional sensors may be used to estimate the values from any failed sensors within a given region. This may be particularly relevant where the adjacent sensors are expected to generate similar force readings.

As can be seen in Table 2 below, the number of row and column connectors in each sensor region do not all match the number of sensors in the regions. However, each sensor region includes at least two unique row connectors and at least two unique column connectors. As a result, if a single connector fails each sensor region still has at least one redundant connector to ensure that the entire region does not fail.

TABLE 2
Arrangement of Sensors Shown in FIGS. 7A and 7B

| | # OF | # OF ROW | # OF COLUMN |
|---|---|---|---|
| | SENSORS | CONNECTORS | CONNECTORS |
| REGION 700a | 5 | 3 | 3 |
| REGION 700b | 4 | 3 | 3 |
| REGION 700c | 4 | 3 | 3 |
| REGION 700d | 4 | 3 | 2 |
| REGION 700e | 3 | 3 | 2 |
| REGION 700f | 6 | 4 | 3 |
| REGION 700g | 6 | 4 | 3 |

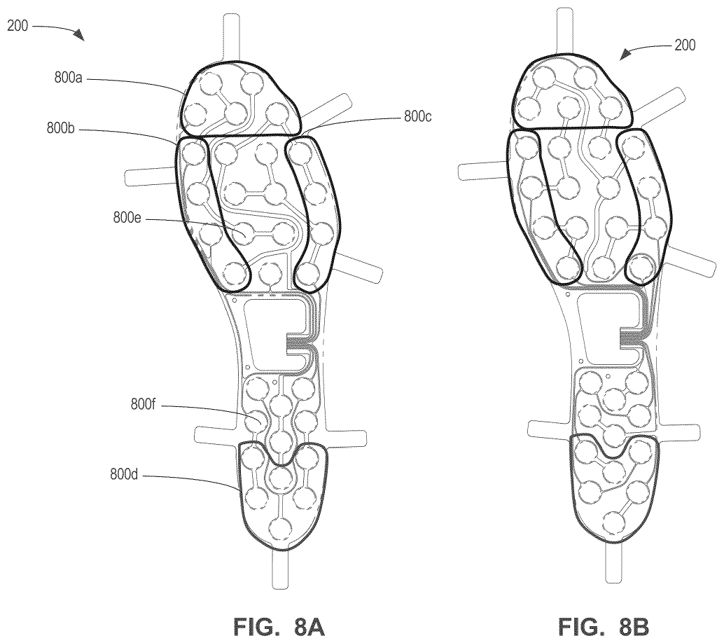

FIGS. 8A and 8B illustrate an example arrangement of sensor regions 800 for the sensing unit 200 shown in FIGS. 2A-2C. FIG. 8A shows the sensing unit 200 with the row traces 205 shown and the column traces 210 omitted. FIG. 8B shows the sensing unit 200 with the column traces 210 shown and the row traces 205 omitted.

As shown in FIGS. 8A and 8B, the sensor regions 800a-800d are multi-sensor regions that include a plurality of region-associated sensors. In addition, there are a plurality of single sensor regions (i.e. regions containing only one sensor) such as sensor regions 800e and 800f. The arrangement of sensor regions shown in FIGS. 8A and 8B allows for different sensor value resolution for different portions of the sensing unit 200. That is, multi-sensor regions (or larger multi-sensor regions) can be selected for those portions of the sensing unit 200 where a lower resolution is sufficient while smaller multi-sensor regions or single sensor regions can be selected for those portions of the sensing unit 200 where higher resolution is required.

The arrangement of sensor regions shown in FIGS. 8A-8B is an example of a sensor region arrangement that may be used as a foot controller for a gaming application. In the example arrangement shown in FIGS. 8A-8B, sensors in the interior portion of the sensing unit 200 are not grouped into multi-sensor regions. However, the sensors in specified locations (toe, heel, medial edge, lateral edge etc.) can be grouped into multi-sensor regions so that a user can activate those regions by applying force anywhere in the region rather than having to activate specific sensors.

In gaming applications, sensors on the outer edge of an insole sensing unit are typically expected to experience the most force. As a result, it may be more difficult to detect forces with the interior sensors of the sensing unit 200. Accordingly, providing the interior sensors as single sensor regions can ensure greater sensitivity in the midfoot region. Inner sensors may also be left ungrouped to identify specific stride sections, such as flat foot (e.g. if all midfoot sensors are activated, this can indicate that a person's foot is flat on the ground).

Alternatively or in addition, the arrangement of sensor regions shown in FIGS. 8A-8B may be used to accommodate variations in foot anatomy/geometry between users. Foot anatomy/geometry varies from user to user. For example, the bony prominence on the edge of the small toes may appear in different locations for different individuals. By decreasing the resolution of the system by generalizing the sensors into regions, users with different bone locations can activate the same region on the sensing system, even if the users technically activate slightly different locations or sensors.

As can be seen in Table 3 below, the number of row and column connectors in each multi-sensor region do not all match the number of sensors in the regions. However, each multi-sensor region includes at least two unique row connectors and at least two unique column connectors. As a result, if a single connector fails each multi-sensor region still has at least one redundant connector to ensure that the entire region does not fail.

TABLE 3
Arrangement of Sensors Shown in FIGS. 8A and 8B

| | # OF | # OF ROW | # OF COLUMN |
|---|---|---|---|
| | SENSORS | CONNECTORS | CONNECTORS |
| REGION 800a | 5 | 3 | 3 |
| REGION 800b | 4 | 3 | 3 |
| REGION 800c | 4 | 2 | 3 |
| REGION 800d | 6 | 4 | 3 |

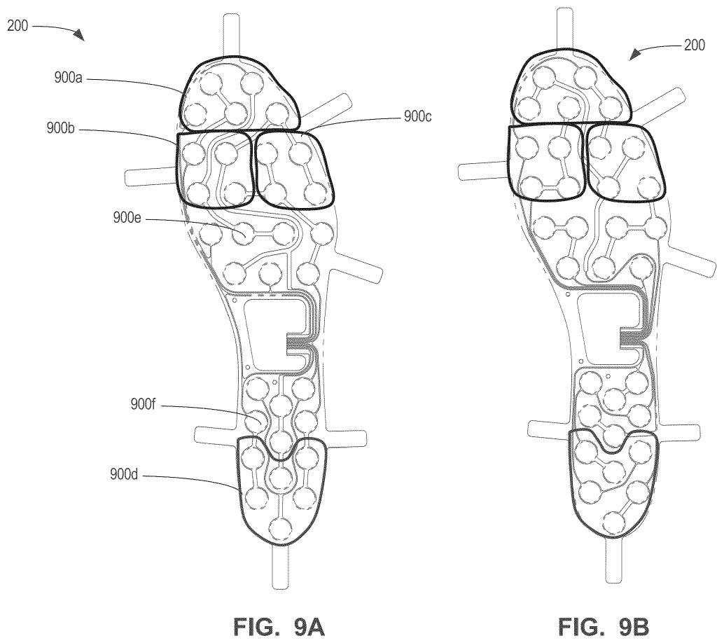

FIGS. 9A and 9B illustrate an example arrangement of sensor regions 900 for the sensing unit 200 shown in FIGS. 2A-2C. FIG. 9A shows the sensing unit 200 with the row traces 205 shown and the column traces 210 omitted. FIG. 9B shows the sensing unit 200 with the column traces 210 shown and the row traces 205 omitted.

As shown in FIGS. 9A and 9B, the sensor regions 900a-900d are multi-sensor regions that include a plurality of region-associated sensors. In addition, there are a plurality of single sensor regions (i.e. regions containing only one sensor) such as sensor regions 900e and 900f. Similar to the arrangement shown in FIGS. 8A and 8B, the arrangement of sensor regions shown in FIGS. 9A and 9B allows for different measurement resolution for different portions of the sensing unit 200.

The arrangement of sensor regions shown in FIGS. 9A-9B is another example of a sensor region arrangement that may be used as a foot controller for a gaming application.

As can be seen in Table 4 below, the number of row and column connectors in each multi-sensor region do not all match the number of sensors in the regions. However, each multi-sensor region includes at least two unique row connectors and at least two unique column connectors. As a result, if a single connector fails each multi-sensor region still has at least one redundant connector to ensure that the entire region does not fail.

TABLE 4
Arrangement of Sensors Shown in FIGS. 9A and 9B

| | # OF | # OF ROW | # OF COLUMN |
|---|---|---|---|
| | SENSORS | CONNECTORS | CONNECTORS |
| REGION 900a | 5 | 3 | 3 |
| REGION 900b | 4 | 4 | 2 |
| REGION 900c | 4 | 2 | 3 |
| REGION 900d | 6 | 4 | 3 |

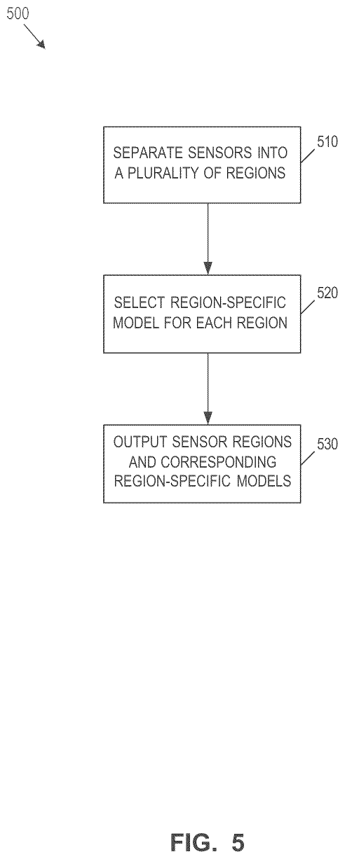

As shown in FIG. 4, step 420 is optional in that method 400 can proceed without an active step of identifying the sensor regions. That is, the sensor regions may be previously identified (e.g. during a preprocessing phase) such that no additional identification is required after the sensor readings are obtained. An example process of grouping the sensors into a plurality of sensor regions is described in further detail herein below with reference to step 510 of method 500 shown in FIG. 5.

A plurality of regional sensor values can then be determined for the plurality of sensor regions. The plurality of regional sensor values can be determined by performing steps 430-440 of method 400 for each sensor region identified at 420.

At 430, a region-specific model can be identified for a particular sensor region. The region-specific model can be defined to specify how the at least one regional sensor value for that sensor region is to be calculated.

Various different types of region-specific models can be used for the sensor regions associated with the plurality of sensors. In some examples, the region-specific model for a given sensor region may be a single-value model. A single-value model is defined to generate a single regional sensor value for the entire sensor region. The single regional sensor value is based on the individual sensor values obtained from the region-associated sensors within that region. The single regional sensor value can be applied over the entire region.

A single-value model can be defined to generate the single regional sensor value in various ways. For example, a single-value model can be defined to generate a single regional sensor value as the sensor value of the most activated region-associated sensor. This may be referred to as a leading sensor activation model. In a leading sensor activation model the single regional sensor value can be determined as the individual sensor value of the most activated sensor in the region (i.e. the region-associated sensor corresponding to the sensor reading with the highest value).

Alternatively, a single-value model can be defined to generate a single regional sensor value as an average sensor value of the at least one region-associated sensor. This may be referred to as an average sensor activation model. In an average sensor activation model the single regional sensor value can be determined as the average of the individual sensor values obtained from the sensors in the region.

Alternatively, a single-value model can be defined to generate a single regional sensor value as a weighted average sensor value of the at least one region-associated sensor. This may be referred to as a weighted average sensor activation model. In a weighted average sensor activation model each sensor can be associated with a corresponding sensor weight (e.g. outer sensors may be given a greater weight than inner sensors) and the single regional sensor value can be determined as the weighted average of the individual sensor values obtained from the sensors in the region.

In some examples, the region-specific model for a given sensor region may be a multi-value model. A multi-value model is defined to generate a plurality of regional sensor values for the sensor region. The plurality of regional sensor values can be applied over the sensor region.

A multi-value model can be defined to generate multiple regional sensor values in various ways. For example, a multi-value model can be defined to determine multiple regional sensor values using estimation techniques such as interpolation and/or extrapolation. This may be the case, for example, where the other sensors in a region are used to interpolate/extrapolate the value of a failed sensor and/or where the sensors in a region are used to estimate sensor values at void locations (e.g. interstitial locations and/or external void locations).

A multi-value model can be defined to estimate multiple regional sensor values using the methods for synthesizing sensor data described in Applicant's co-pending patent application Ser. No. 17/988,468 filed on Nov. 16, 2022 entitled “SYSTEM AND METHOD FOR SYNTHESIZING SENSOR READINGS”, and/or the methods for synthesizing sensor data described in Applicant's co-pending patent application Ser. No. 18/183,642 filed on Mar. 14, 2023 entitled “SYSTEM AND METHOD FOR DETERMINING USER-SPECIFIC ESTIMATION WEIGHTS FOR SYNTHESIZING SENSOR READINGS”.

Alternatively, a multi-value model can be defined to determine multiple regional sensor values by applying a function or equation to the corresponding sensor region. The multi-value model can define a region-specific function (e.g. a sine function) used to generate the plurality of regional sensor values based on the sensor readings from the region-associated sensors.

As shown in FIG. 4, step 430 is optional in that method 400 can proceed without an active step of identifying a region-specific model. That is, the region-specific models associated with each sensor region may be previously identified (e.g. during a preprocessing phase) such that no additional identification is required after the sensor readings are obtained. An example process for determining the region-specific model to be applied to a given region is described in further detail herein below with reference to step 520 of method 500 shown in FIG. 5.

At 440, the at least one regional sensor value for a given sensor region can be generated by applying the region-specific model (e.g. from 430) to the sensor readings obtained at 410 from the at least one region-associated sensor corresponding to that sensor region.

It should be understood that different region-specific models can be used for different sensor regions in the same sensing unit. In some examples, both single-value and multi-value models may be used for different regions of the same sensing unit.

Optionally, the region-specific models can be used in conjunction with the methods of synthesizing sensor data at void locations described in U.S. patent application Ser. Nos. 17/988,468 and 18/183,642 referenced herein above. For example, the region-specific models can be defined to estimate regional sensor values to address conductivity or sensor failures. These estimated regional sensor values can then be used with the methods described in U.S. patent application Ser. Nos. 17/988,468 and 18/183,642 to determine estimated sensor values at sensor void locations.

The region-specific models can be applied to the corresponding sensor readings in real-time and/or non-real-time. For example, region-specific models may be applied to the sensor readings in real-time for applications such as gaming that require data to be processed in real-time in order to participate in the activity. Alternatively, region-specific models may be applied to the sensor readings in non-real-time or post-data collection (e.g. at a later time) to analyze the user's biomechanics and movement for various purposes such as injury monitoring.

In some cases, it may be computationally expensive to compute all of the regional sensor values onboard the sensing unit 102. Accordingly, some of the regional sensor values may be determined by a remote processing device 108 and/or cloud server 110. However, this can also introduce challenges in that the sensor data collected by the sensing unit 102 needs to be transmitted to the remote processing device 108 and/or cloud server 110 to allow the regional sensor value(s) to be determined. This can be problematic, particularly for real-time applications, if the volume of sensor data is large. Accordingly, it may be desirable to reduce the volume of data that needs to be transmitted by the sensing unit 102.

As noted above, the region-specific model can be defined as a region-specific function or equation. In order to determine the at least one regional sensor value using the region-specific function the coefficients for the region-specific function need to be determined from the sensor measurements received at 410.

Optionally, coefficient values for the region-specific model or equation can be calculated onboard the sensing unit and transmitted to an external processing device. This may allow the at least one regional sensor value to be determined without transmitting all of the sensor measurements received at 410 to the remote processing device 108 and/or cloud server 110.

For a given sensor region, the number and location of sensors as well as the overall area of the sensor region can be known in advance. The general characteristics of the sensor data for that sensor region may also be known in advance for various different events during a gait cycle. A region-specific function can then be defined to represent the regional sensor value(s) for that region. The coefficients for the region-specific function can be calculated from the sensor measurements (received at 410) onboard the sensing unit 102 and transmitted to the remote processing device 108 and/or cloud server 110. This can reduce the volume of data that needs to be transmitted by the sensing unit 102 and reduce the communication bandwidth required.

As a simplified example, a hindfoot region may be defined with six individual sensors. Accordingly, six sensor measurements for the hindfoot region would be received on a continual basis. The general shape of the force sensor data in the hindfoot region for a particular event (e.g. a heel strike event) can be known in advance (e.g. a 3D peak). The region-specific function for the hindfoot region can be defined as force=Acos(B²+C²)+D where A, B, C and D are the coefficients of the region-specific function. Accordingly, the sensing unit can calculate the four coefficients of the region-specific function onboard and transmit the coefficient values to the remote processing device 108 and/or cloud server 110. As a result, the regional sensor value can be determined with the transmission of four coefficient values instead of six sensor measurement values thereby reducing the bandwidth requirements for the system.

Optionally, different region-specific models (and even different sensor region groupings) may be applied to the same set of sensor readings. For example, a first combination of sensor regions and region-specific models may be applied in real-time to allow a user to participate in a gaming activity. Subsequently, a different combination of sensor regions and region-specific models may be applied to the same sensor readings for use with a different application (e.g. evaluating the user's athletic performance or monitoring for a risk of injury).

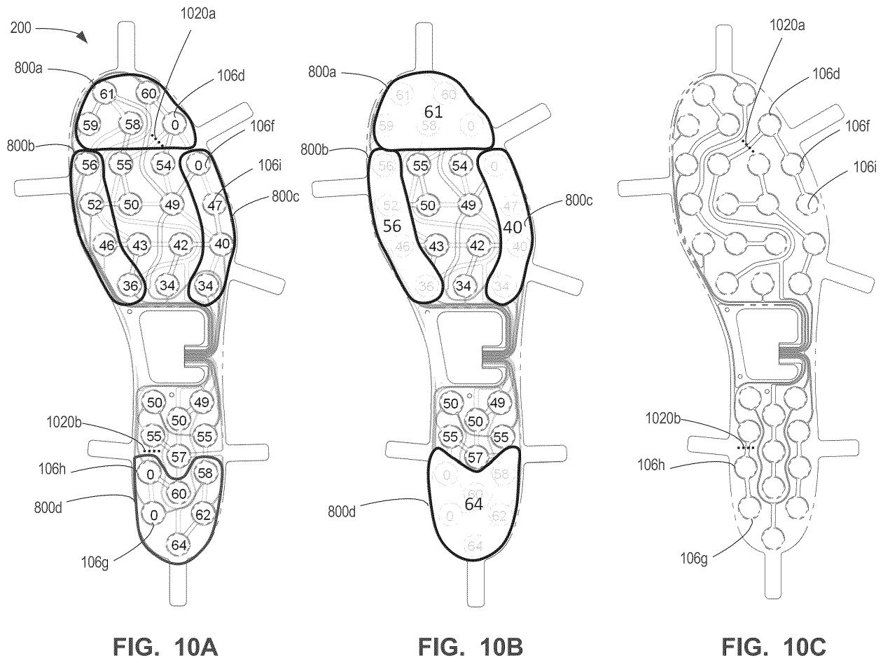

FIGS. 10A-10C illustrate an example of the regional sensor values generated using an example arrangement of sensor regions (corresponding to the arrangement shown in FIG. 8A-8B). FIGS. 10A and 10B show the sensing unit 200 with both the row traces 205 and the column traces 210 shown. FIG. 10C shows the sensing unit 200 with the row traces 205 shown and the column traces 210 omitted.

FIG. 10A shows the individual sensor readings obtained from the sensors in sensing unit 200. As shown in FIGS. 10A and 10C, conductivity failures 1020a and 1020b are present in the sensing unit 200 and sensors 106d, 106f, 106g and 106h are no longer connected to the electronics module 104. As a result, the sensor readings from sensors 106d, 106f, 106g and 106h show null individual sensor values (see FIGS. 10A and 10B).

In addition, sensor 106i shows a non-zero individual sensor value. However, because sensor 106i is downstream from the conductivity failure 1020a it has been identified as a sensor expected to fail.

FIG. 10B shows the regional sensor values generated from the individual sensor values shown in FIG. 10A. In the example shown in FIG. 10B, a leading sensor activation model was selected as the region-specific model for multi-sensor regions 800a-800d. Accordingly, the regional sensor value for each region 800a-800d is calculated as the largest individual sensor value from a functioning sensor (i.e. omitting sensor 106i that is considered a failed sensor) in that region as shown in FIG. 10B.

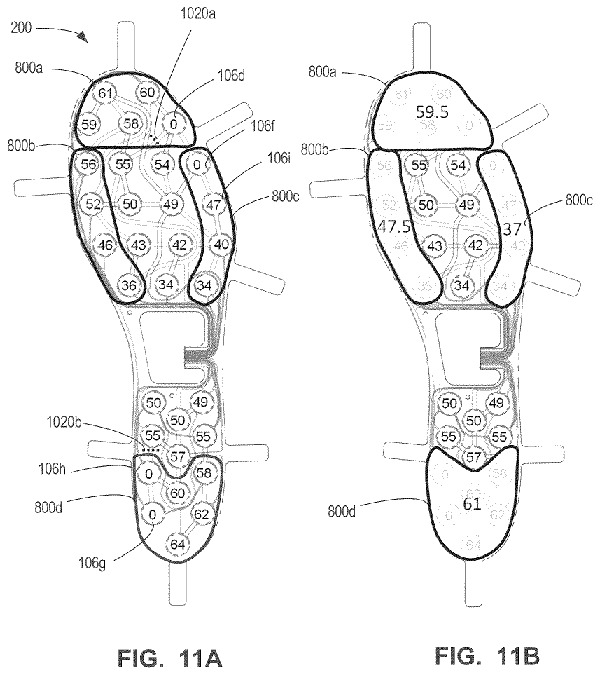

FIGS. 11A-11B illustrate another example of the regional sensor values generated using an example arrangement of sensor regions (corresponding to the arrangement shown in FIG. 8A-8B). FIGS. 11A and 11B show the sensing unit 200 with both the row traces 205 and the column traces 210 shown.

FIG. 11A shows the individual sensor reading obtained from the sensors in sensing unit 200. As with the sensing unit 200 shown in FIG. 11A, conductivity failures 1020a and 1020b are again present in the sensing unit 200. Accordingly sensors 106d, 106f, 106g, 106h and 106i have been identified as failed sensors.

FIG. 11B shows the regional sensor values generated from the individual sensor values shown in FIG. 11A. In the example shown in FIG. 11B, an average sensor activation model was selected as the region-specific model for multi-sensor regions 800a-800d. Accordingly, the regional sensor value for each region 800a-800d is calculated as the average of the individual sensor values from the functioning sensors in that region as shown in FIG. 11B.

Optionally, the sensor value for one or more sensors may be adjusted based on a relative level of activation of one or more adjacent sensors. The adjusted sensor values can then be provided as inputs to the region-specific model at 440.

This adjustment (also referred to as an activation multiplier) may be applied to sensors that tend to have a lower degree of activation as compared to adjacent sensors. This can allow the less active sensors to achieve the same degree of activation as sensors with higher activation (e.g. increasing the sensitivity of the less active sensors). For example, the values from the less active sensors may be increased by an activation multiplier value so that they are within the same range as the values measured by the more active sensors.

For example, the interior sensors of the sensor arrangement shown in FIGS. 8 and 9 may be expected to experience less activation in a gaming application. Accordingly, an activation multiplier may be applied to these interior sensors to increase the values measured by these sensors, such that they are in the same ranges as the values measured by the outer sensors.

The value of an activation multiplier can be determined in various ways. For example, a ratio between the average activation of a less active sensor (e.g. the inner sensors in FIGS. 8 and 9) and a more active sensor (e.g. the outer sensors in FIGS. 8 and 9) may be calculated. The activation multiplier value can then be determined based on the ratio.

At 450, the plurality of regional sensor values generated at 440 can be output. The regional sensor values can be output directly through an output device to provide an individual with feedback on the activity being monitored. Optionally, the sensor readings may also be output. Alternately or in addition, the sensor readings and/or regional sensor values may be stored, e.g. for later review, comparison, analysis, or monitoring.

Optionally, additional parameters or derived data values can be determined from the regional sensor values. These additional parameters may also be output to an individual as feedback, to a storage device, or to an analysis device or applications. For example, the additional parameters may include force derivative values or force-based metrics calculated from the regional sensor values.

As noted above, the sensors may be force sensors mapped to specific locations of an insole. Accordingly, at 450, the controller may output a force grid that includes the regional sensor values determined at 440 (e.g. as shown in FIGS. 10B and 11B). The force grid can identify regional force values determined for all regions of the insole, including regions with a failed sensor.

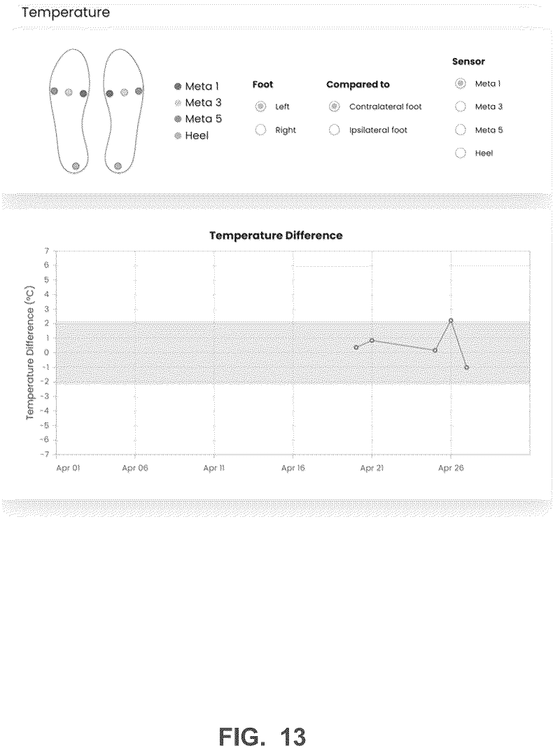

Optionally, the regional sensor values may be provided to the user or to other individuals who may use the regional sensor values to assist the individual (e.g. for patient monitoring). FIG. 13 illustrates an example of a dashboard that may be displayed to a user or other individual. The dashboard can include regional force sensor values, regional temperature sensor values and/or other data derived therefrom.

The dashboard may be controllable by a user to display regional sensor values for selected sensor regions. For example, the plurality of sensor regions may be defined according to general anatomical regions of the foot (e.g. metatarsal 1, metatarsal 3, metatarsal 5, heel 4, etc. as shown in FIG. 13). A user may select the sensor regions to be displayed in the dashboard to allow for review of regional sensor values of interest.

As shown, the dashboard may also allow a user to compare regional sensor values in various ways, e.g. over time (e.g. for the same foot or both feet), contralaterally (e.g. compared to the same region(s) on the opposite foot), ipsilaterally (compared to the overall average of the same foot), and so forth. The dashboard can also display the regional sensor values in various ways, e.g. as tables, graphs, mappings etc.

Referring now to FIG. 5, shown therein is an example method 500 of determining sensor regions and region-specific models, which may be used to determine regional sensor data, for example using method 400.

Method 500 can be implemented as a preprocessing method that can be used to determine an arrangement of sensor regions and region-specific models for a sensing unit. The method 500 may be performed initially for a sensor array (e.g. when the sensor array is manufactured or when the user updates the software or firmware associated with the sensor array). The sensor regions and region-specific models generated through method 500 can be stored for subsequent use in determining regional sensor data for a user when they are using the corresponding sensor array.

Alternatively or in addition, method 500 may be repeated during use of the sensor array (e.g. when the sensing unit is being used for a new use and/or in response to a sensor failure).

Alternatively or in addition, method 500 may be repeated multiple times during an initial preprocessing phase to determine potential arrangements of sensor regions and region-specific models for different applications and/or different sensor operational conditions. The potential sensor regions and region-specific models generated through method 500 can be stored for subsequent use in determining regional sensor data for a user when they are using the corresponding sensor array for the corresponding application or operational conditions. This may simplify the process of modifying the sensor regions and/or region-specific models in response to changes in the activity being performed or in response to sensor failures.

At 510, the plurality of sensors can be separated into a plurality of distinct regions or portions. As explained above with reference to FIGS. 6-9, the sensors can be grouped into a plurality of sensor regions.

The sensor regions typically include at least one multi-sensor region. Each multi-sensor region includes a plurality of sensors.

Optionally, the sensor regions may also include at least one single sensor region. Each single sensor region only includes one sensor.

The grouping of the sensors into sensor regions can vary depending on the purpose of the sensor grouping. In some examples, the sensor regions can be defined to compensate for conductivity failures in a sensing unit. Alternatively or in addition, the sensor regions can be defined to adjust (i.e. increase or decrease) the measurement resolution of the sensing unit.

To compensate for conductivity failures in a sensing unit, the sensor regions can be defined to include redundant row connectors and/or column connectors. For example, for each multi-sensor region the plurality of region-associated sensors can be connected to at least two different row connectors and at least two different column connectors. This is the case for each of the multi-sensor regions shown in FIGS. 6-9.

To maximize connector redundancy in order to compensate for conductivity failures, the sensor regions can be defined so that each region-associated sensor within a region has a unique row connector and column connector. For example, for each multi-sensor region the plurality of region-associated sensors can be connected to a different row connector and column connector from every other region-associated sensor within that multi-sensor region. This is the case for the multi-sensor regions 600 shown in FIG. 6. This can ensure that in the event of a connector conductivity failure, where all sensors downstream of the broken connector would fail, only a single sensor would be lost in each region containing a sensor associated with the failed row/column connector.

Alternatively or in addition, the sensor regions can be defined to group adjacent sensors. Grouping sensor adjacently can ensure that sensor regions contain sensors expected to experience similar force values. This can be beneficial where the values from the functioning sensors may be used to determine regional sensor values in regions with failed sensors.

The sensor regions can also be defined to balance multiple factors (e.g. grouping adjacent sensors while ensuring that the sensor regions contain multiple row and column connectors as shown in FIG. 7A-7B).

Alternatively, the sensor regions can be defined to group sensors that are not adjacent (e.g. a sensor in the toe and a sensor in the heel could be grouped in the same region). This may ensure that the sensors in a region each have a unique row connector and a unique column connector.

Grouping non-adjacent sensors may also be useful in identifying certain portions of a user's gait cycle. For example, a sensor region that includes a sensor in the toe and a sensor in the heel may be used to verify a foot flat time for the user (e.g. where force is present on both the user's toe and heel) and/or a swing phase for the user (e.g. where no force is present).

Alternatively or in addition, the sensor regions can be defined based on a desired resolution of the sensing unit or a portion of the sensor unit. The number of sensors within a given sensor region can be selected based on the desired resolution (e.g. larger sensor regions corresponding to a lower resolution/reduced granularity). This may reduce the computation requirements involved in determining the regional sensor values.

Optionally, the plurality of sensor regions can include sensor regions of varied size. For example, the plurality of sensor regions may include both multi-sensor regions and single sensor regions. This may allow different portions of the sensing unit to generate regional sensor values with different levels of resolution (see e.g. FIGS. 8 and 9).

Even when grouping sensors based on resolution, it may still be desirable to account for potential conductivity failures. Accordingly, the multi-sensor regions may still be defined to include multiple row connectors and multiple column connectors to reduce the risk of a complete region failure.

Optionally, the plurality of sensor regions can include at least two sensor regions that at least partially overlap. Overlapping sensor regions can refer to sensor regions where at least one sensor is contained within both sensor regions.

Overlapping sensor regions may be useful in a variety of scenarios. Overlapping sensor regions may help smooth transitions/reduce differences in the regional sensor value(s) determined for adjacent or nearby sensor regions. For example, a first region may include forefoot sensors while a second sensor region includes mid-foot sensors. There may be a large difference in the regional sensor values for these regions, even if adjacent, due to the shape of an individual's arch. Providing an overlap between the two regions can help reduce the difference in the regional sensor values for these adjacent regions. Overlapping sensor regions may also provide a balance between fast response times (e.g. required for gaming applications) and data accuracy (e.g. useful for data modelling for applications such as injury prevention).

The sensors can be grouped into the sensor regions manually. Alternatively, the sensors may be grouped into sensor regions automatically. For example, an optimization algorithm can be used to determine the arrangement of regions based on one or more factors (e.g. desired resolution, reduced risk of failure, grouping adjacent sensors etc.).

At 520, a region-specific model can be selected for each sensor region identified at 510. Various examples of region-specific models are described herein above at 430 in method 400. The selection of the region-specific models may depend on the intended use of the sensing unit.

Single-value models may be desirable for applications where high resolution sensor values are not required. Accordingly, a single regional value may be sufficient to represent an entire region. However, multi-value models may also be used for applications involving a low resolution requirement (e.g. to reduce the impact of conductivity failures).

Multi-value models may be desirable to minimize the impact of conductivity failures. Multi-value models may allow the functional sensors in a region to estimate the value of failed sensors within the region. However, single value models may also be used in circumstances where it is desirable to minimize the impact of conductivity failures, e.g. where it is also desirable to reduce the computational cost of processing the sensor readings.

Optionally, the same model or same type of model may be applied to all of the sensor regions in a sensor array. Alternatively, different models may be selected for different regions within a single sensing unit. For example, leading sensor activation may be applied to a toe region, while interpolation is applied to a heel region. The model that is applied to a certain region may also change with time/circumstances. For example, leading sensor activation may be applied to a region when all sensors are functioning, but if one of the sensors fails, an interpolation model may be applied.

At 530, the sensor regions and region-specific models can be output. The sensor regions and region-specific models can be output for storage in a non-transitory storage medium of a device such as the input unit 102, processing device 108 and/or cloud server 110. The sensor regions and region-specific models can subsequently be accessed and used to analyze sensor readings obtained for the user (e.g. as part of a method for determining regional sensor data such as method 400).

Optionally, outputting the sensor regions and region-specific models can include storing the sensor regions and region-specific models locally in a non-transitory storage module of a wearable device or fitness equipment containing the plurality of sensors. This can allow the regional sensor data to be calculated for a user in real-time. For example, the sensor regions and region-specific models can be loaded onto the firmware of a sensing unit such as sensing unit 200 during a firmware update.

Alternatively or in addition, the sensor regions and region-specific models can be stored remotely from the wearable device or fitness equipment containing the plurality of sensors. The sensor regions and region-specific models can then be accessed when analyzing sensor readings obtained for the user.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A method for determining regional sensor data, the method comprising:
obtaining a plurality of sensor readings from a corresponding plurality of sensors, the plurality of sensors arranged in a first predetermined pattern, wherein the first predetermined pattern maps each of the plurality of sensors to respective locations on a carrier device and the plurality of sensors are grouped into a plurality of sensor regions, wherein each sensor region includes at least one region-associated sensor from the plurality of sensors; and
determining a plurality of regional sensor values for the plurality of sensor regions by, for each sensor region:
identifying a region-specific model, the region-specific model specifying how the at least one regional sensor value for that sensor region is to be calculated; and
generating the at least one regional sensor value for that sensor region by applying the region-specific model to the sensor readings obtained from the at least one region-associated sensor corresponding to that sensor region.

2. The method of claim 1, further comprising:
identifying one or more failed sensors in the plurality of sensors; and
for each failed sensor, omitting the sensor readings corresponding to that failed sensor from the step of generating the regional sensor value for the corresponding sensor region.

3. The method of claim 1, wherein the plurality of sensors are grouped into the plurality of sensor regions based on a desired resolution, and the number of region-associated sensors within each sensor region is determined at least in part based on the desired resolution.

4. The method of claim 1, wherein for at least one of the sensor regions the region-specific model is defined as a single-value model configured to generate a single regional sensor value for the entire sensor region.

5. The method of claim 4, wherein the single regional sensor value is determined as one of a sensor value of the most activated region-associated sensor, an average sensor value of the at least one region-associated sensor, and a weighted-average sensor value of the at least one region-associated sensor.

6. The method of claim 1, wherein the sensor value for a particular sensor is adjusted based on a relative level of activation of one or more adjacent sensors.

7. The method of claim 1, wherein for at least one of the sensor regions the region-specific model is defined as a multi-value model configured to generate a plurality of regional sensor values for the sensor region.

8. The method of claim 7, wherein the multi-value model comprises one of an interpolation model and an extrapolation model.

9. A system for determining regional sensor data, the system comprising:
a plurality of sensors arranged in a first predetermined pattern, with each of the plurality of sensors arranged at respective locations on a carrier device, wherein the plurality of sensors are associated with a plurality of sensor regions, wherein each sensor region includes at least one region-associated sensor from the plurality of sensors; and
one or more controllers communicatively coupled to the plurality of sensors, the one or more controllers configured to:
obtain a corresponding plurality of sensor readings from the plurality of sensors;
determine a plurality of regional sensor values for the plurality of sensor regions by, for each sensor region:
identify a region-specific model, the region-specific model specifying how the at least one regional sensor value for that sensor region is to be calculated; and
generate the at least one regional sensor value for that sensor region by applying the region-specific model to the sensor readings obtained from the at least one region-associated sensor corresponding to that sensor region.

10. The system of claim 9, wherein the one or more controllers is further configured to:
identify one or more failed sensors in the plurality of sensors; and
for each failed sensor, omit the sensor readings corresponding to that failed sensor from the step of generating the regional sensor value for the corresponding sensor region.

11. The system of claim 9, wherein the plurality of sensor regions includes at least one multi-sensor region, each multi-sensor region including a plurality of region-associated sensors.

12. The system of claim 11, wherein the plurality of sensors are electrically connected to an electronics module using a plurality of electrical connectors, the plurality of electrical connectors including a plurality of row connectors and a plurality of column connectors, wherein each sensor is electrically connected to a pair of electrical connectors in the plurality of electrical connectors, the pair of electrical connectors connected to each sensor includes one row connector and one column connector, and the pair of electrical connectors connected to each sensor is unique.

13. The system of claim 12, wherein for each multi-sensor region the plurality of region-associated sensors are connected to at least two different row connectors and at least two different column connectors.

14. The system of claim 13, wherein for each multi-sensor region, each region-associated sensor is electrically connected to a different row connector and column connector from every other region-associated sensor within that multi-sensor region.

15. The system of claim 9, wherein for at least one of the sensor regions the region-specific model is defined as a single-value model configured to generate a single regional sensor value for the entire sensor region.

16. The system of claim 15, wherein the single regional sensor value is determined as one of a sensor value of the most activated region-associated sensor, an average sensor value of the at least one region-associated sensor, and a weighted-average sensor value of the at least one region-associated sensor.

17. The system of claim 9, wherein the one or more controllers is further configured to adjust the sensor value for a particular sensor based on a relative level of activation of one or more adjacent sensors.

18. The system of claim 9, wherein for at least one of the sensor regions the region-specific model is defined as a multi-value model configured to generate a plurality of regional sensor values for the sensor region, wherein the multi-value model comprises one of an interpolation model and an extrapolation model.

19. The system of claim 9, further comprising the carrier device wherein the carrier device is a wearable device and the plurality of sensors are provided by the wearable device, and the wearable device is an insole worn on a foot.

20. The system of claim 9, wherein the plurality of sensors are force sensors.
